(19) United States
(12) Patent Application Publication
(10) Pub. No.: US 2025/0204758 A1
(43) Pub. Date: Jun. 26, 2025

(54) Articulation link for or in a deflection mechanism

(71) Applicant: KARL STORZ SE & Co. KG, Tuttlingen (DE)

(72) Inventors: Leonik Lander, Dietingen (DE); Alexander Fuchs, Mühlheim (DE)

(21) Appl. No.: 18/987,547

(22) Filed: Dec. 19, 2024

(30) Foreign Application Priority Data
Dec. 20, 2023 (DE) 102023135983.5

(51) Int. Cl. A61B 1/008; A61B 17/00; A61M 25/01

(52) U.S. Cl. CPC A61B 1/008; A61B 17/00234; A61B 2017/00314; A61M 25/0138

(57) ABSTRACT

The present invention relates to an articulation member for or in a deflection mechanism at a distal end of a surgical instrument, in particular an endoscope, with a base body which has an axial through-opening along a longitudinal axis so that a partial portion of a working channel of the surgical instrument can be formed with the through-opening or can be guided through the through-opening, and at least one first connecting portion which is arranged on an axial first end portion of the base body, wherein the connecting portion has an extension at least partially aligned in a radial direction of the base body which is designed to be connected to a connecting portion of a further articulation member so that a keyed and articulated connection can be formed between two articulation members. The present invention further relates to a deflection mechanism and a method for producing a deflection mechanism.

### FIELD OF THE INVENTION

This application claims priority to German Patent Application No. 10 2023 135 983.5 filed Dec. 20, 2023, and entitled “Artikulationsglied für eine oder in einer Auslenkmechanik,” which is incorporated herein by reference.

### FIELD OF THE INVENTION

The present invention relates to an articulation member for a or in a deflection mechanism at a distal end of a surgical instrument. Furthermore, the invention relates to a deflection mechanism and a method for producing a deflection mechanism.

### BACKGROUND

A deflection mechanism for endoscopic instruments serves to bend a tool or accessory attached thereto, in particular forceps, scissors, clamp, laser fiber or similar. The deflection mechanism therefore serves, on the one hand, to align the tool at a distal end of the surgical instrument such as an endoscope. Furthermore, an accessory for imaging, lighting and/or a steering assembly can be guided in particular by steering means such as pull wires or similar with the aid of the deflection mechanism.

The deflection mechanism can also be referred to as a bending unit or articulation unit which has a through-opening with a working channel through which the accessory is guided. The accessory, such as forceps, scissors, clamp, laser fiber or similar, is usually pushed through the working channel of the endoscopic instrument. In addition to this working channel, a light and/or imaging unit can be permanently installed in the endoscopic instrument.

In order to allow for bending, the deflection mechanism is made of individual articulation members that are articulated to one another. The deflection or alignment of the deflection mechanism can be carried out with pull wires which are also guided at least partially through the through-opening, in particular in separate openings.

Due to the large number of possible accessories as well as current and light-conducting components, the through-opening should be as spacious as possible and free of obstacles. However, the connection of the individual articulation members can lead to cross-sectional constrictions in the interior, which sectionally reduce the cross section of the through-opening. This is the case, for example, when the individual articulation members are connected to one another via connecting means such as rivets, bolts or pins, wherein the connecting means protrude into the cross section of the through-opening and in particular represent angular obstacles. Furthermore, the connecting means must be firmly connected to one of the articulation members so that it is stored securely. This requires additional effort and associated time and costs.

To avoid this, a deflection mechanism is known from EP 1 604 607 A1, wherein hinge-shaped connections are formed between individual articulation members, wherein the individual articulation members are connected via a type of movable dovetail connection. A pin is aligned in the longitudinal direction of the articulation member and engages in a recess of an adjacent articulation member. The recess has a semicircular shape so that a rotation of the articulation members relative to one another is made possible. However, such a connection requires high demands on dimensional accuracy and precision, since the hinge joint is only designed to be the thickness of the wall thickness of the articulation member, and the articulating mechanism itself already has a very small cross section. Furthermore, the maximum rotational movement of the two members is limited by the size of the semicircular recess. Particularly large angles can hardly be implemented because the remaining material thickness between the ends of the semicircular recess becomes very small as the circumference increases.

### SUMMARY

Against this background, the present invention is based on the object of providing an improved articulation member and an improved deflection mechanism.

According to the invention, this object is achieved by an articulation member having the features of claim 1, a deflection mechanism having the features of claim 11, and a method having the features of claim 16.

Accordingly, the following are provided:

An articulation member for or in a deflection mechanism at a distal end of a surgical instrument, in particular an endoscope, with a base body which has an axial through-opening along a longitudinal axis so that a partial portion of a working channel of the surgical instrument can be formed with the through-opening or can be guided through the through-opening, and at least one first connecting portion which is arranged on an axial first end portion of the base body, wherein the connecting portion has an extension at least partially aligned in a radial direction of the base body which is designed to be connected to a connecting portion of a further articulation member so that a keyed and articulated connection can be formed between two articulation members.

A deflection mechanism for alignment, in particular for tool alignment and/or for alignment of a lighting, rinsing and/or an imaging unit, at a distal end of a surgical instrument, in particular an endoscope, with a plurality of articulation members, wherein the articulation members are connected to one another in a keyed and articulated manner such that an articulated working channel is formed through the through-openings of the surgical instrument, or a flexible working channel can be passed through the through-openings.

A method for producing a deflection mechanism for alignment, in particular for tool alignment and/or for alignment of a lighting, rinsing and/or an imaging unit, at a distal end of a surgical instrument, in particular an endoscope, comprising the steps of: providing a plurality of articulation members, at least partially pushing adjacent articulation members into one another, wherein connecting portions of adjacent articulation members are displaced over one another until an extension of a first connecting portion at least partially aligned in a radial direction of the base body engages in a connecting portion of a second articulation member so that a keyed and articulated connection of the articulation members is produced.

The idea underlying the present invention is to create a joint connection of individual articulation members, which is possible solely from the geometry of the articulation members themselves, i.e., in particular a geometry of axial end portions or edge regions of the articulation members.

The base body is designed in particular as a tubular or sleeve-shaped element which is open in particular at both end portions. This allows an axial through-opening to be formed along the longitudinal axis of the articulation member. The end portions preferably have a circular or elliptical cross section. Other cross sections are also conceivable.

A connecting portion is arranged on at least one end portion and has an extension that is at least partially aligned in a radial direction of the base body. In particular, the extension can be aligned in the radial direction so that a rotation axis or a joint axis can be formed which runs orthogonal to a longitudinal axis of the base body. Accordingly, the alignment of the extension can influence the orientation of the joint axis.

The extension can, for example, replace a rivet or a bolt, wherein the extension is advantageously designed integrally connected to the base body, for example as a dome. As a result, the extension is securely mounted on the base body from the outset which means that work steps can be saved when assembling a deflection mechanism.

The extension of the first connecting portion engages with a connecting portion of a second articulation member. The connecting portions of adjacent articulation members are in particular designed complementary with one another so that the two connecting portions can anchor with one another or hook onto one another. By threading the second connecting portion into the first connecting portion, a joint connection can be formed which allows for rotation of adjacent articulation members relative to one another. A keyed and articulated connection between two articulation members allows for the articulation members to be pivoted or rotated relative to one another. A rotation axis is defined in particular by the alignment of the extension.

An extension is to be understood in particular as a type of pin or hollow pin or a dome which in particular has a round cross section. This can allow for a rotation of two articulation members relative to one another, wherein a connecting portion of an adjacent articulation member can rotate around the extension. The extension is provided in particular on an outer surface of the base body and is aligned in particular outwards in the radial direction. Likewise, the extension can be provided on an inner surface of the base body and, in particular, can be aligned inwards in the radial direction.

The articulation member is designed for or in a deflection mechanism of a surgical instrument, wherein any accessory such as in particular a working channel for a tool such as forceps, scissors, laser fiber, collecting basket or similar can be deflected or guided by means of the deflection mechanism. Furthermore, imaging, lighting and/or steering, in particular by pull wires, as well as a rinsing, suction and/or working channel can be guided with the aid of the deflection mechanism.

The plurality of accessories must be guided through the through-opening of the articulation member or the deflection mechanism. It is advantageous if the inner diameter of the through-opening is, on the one hand, designed continuously constant and, on the other hand, as free of “obstacles” as possible. In particular in the case of very small diameters of the articulation members such as diameters in the millimeter range, preferably less than or equal to 3 mm, high demands are therefore placed on the joint connection between adjacent articulation members so that these should be designed to save space. The proposed keyed and articulated connection offers the advantage that it hardly, or in particular never, engages in the inner cross section of the base element. In particular, a protrusion of the connecting portion into the interior of the base element is so small that the working channel is not disturbed. The articulation member therefore offers advantages in particular for particularly small diameters of the base body, in particular in the millimeter range, preferably for diameters of 3 mm or smaller.

The surgical instrument can in particular be an endoscopic instrument, preferably an endoscope. The articulation member is also suitable for other surgical instruments where miniaturization of the deflection mechanism is advantageous.

Advantageously, a rivet-free connection between articulation members can therefore be achieved, which can in particular be achieved solely from a keyed connection between axial end portions. The articulation members can be simply plugged together, wherein a joint connection can be formed by resilient deformation of at least one connecting portion. In this way, the structure of articulation members according to the invention is in particular suitable for the one-time use surgical instruments (so-called “single-use” instruments).

For example, so-called end members can be used at the end portions of the deflection mechanism which have a connecting portion only at one end portion and are formed without a connecting portion at the other end portion.

Advantageous embodiments and developments are shown in the further dependent claims and in the description with reference to the figures in the drawing.

In a preferred embodiment, a second connecting portion can be arranged at an axial second end portion opposite the first end portion, wherein the second connecting portion is designed resiliently so that the keyed connection to a connecting portion of a further articulation member can be established by a resilient deflection. Resilient means in particular that the connecting portion can be resiliently deflected in two opposite directions so that the connecting portion resiliently deforms back to its original position when it is connected to the connecting portion of an adjacent articulation member. This allows the second connecting portion to at least partially encompass the extension. The articulation member is therefore preferably formed from a metal and can in particular be made by being cut out of a tube.

In a preferred embodiment, the second connecting portion can be resiliently deflectable transversely to the radial direction and/or in the radial direction. This allows one connecting portion to be pushed over another connecting portion and thereby deforming resiliently back and forth. If, for example, the second connecting portion is to be pushed over the extension in order to then anchor itself to the extension, a resilient deflection in the radial direction, which is designed to be self-resetting, is advantageous. If the second connecting portion is to be pushed by next to the extension, i.e., in particular to the side of the extension, in order to then encompass the extension in the manner of a clamp or fork, it is advantageous if the second connecting portion is designed to be resiliently transverse to the radial direction, i.e., in particular in a tangential direction.

In a preferred embodiment, the second connecting portion can have a guide contour which is arranged on the connecting portion at an end facing away from the base body and allows for a guided resilient deformation of the second connecting portion in the radial direction. The guide contour can be formed as a type of guide tab or wing which has a slope so that the second connecting portion can be lifted more easily via the extension when two articulation members are connected to one another.

In a preferred embodiment, the second connecting portion can be designed to be fork-shaped, wherein the keyed and articulated connection can be formed by encompassing the extension of a further articulation member. Fork-shaped means in particular that the second connecting portion has two legs which are in particular mirrored to one another. With the two legs, the second connecting portion can encompass the extension. If the legs are resiliently formed, in particular transverse to the radial direction, they can resiliently deform back against the extension when they engage or snap into place around the extension. In particular, the recess between the two legs has an undercut so that the extension can be held in the undercut when the two articulation members are connected to one another. This can make it more difficult to release the extension from the second connecting portion, although a rotational movement remains possible.

In a preferred embodiment, the second connecting portion can have a recess complementary to the extension. This allows an aligned and centered positioning of the articulation members which allows a defined deflection mechanism to be implemented.

In a preferred embodiment, two first connecting portions can be provided at the first end portion. In a preferred embodiment, they can be formed in particular opposite one another on a cross section of the first end portion and/or mirrored to one another. For example, a defined joint axis can be implemented between the two connecting portions.

In a preferred embodiment, two second connecting portions can be provided on the second end portion, which are in particular formed opposite one another on a cross section of the second end portion and/or mirrored to one another. For example, a defined joint axis can be implemented between the two connecting portions.

In a preferred embodiment, the extension can be designed for articulated engagement with a second connecting portion of a further articulation member on a predetermined joint axis. In particular, the joint axis can run through the center of the base body. In contrast to a rivet connection or a bolt, the joint axis can be formed directly by a part of the articulation member itself.

In a preferred embodiment of the deflection mechanism, the connecting portions of adjacent articulation members can have a complementary shape. This allows a joint axis to be formed around which the two articulation members can be moved in any rotational manner relative to one another. The angle of rotation is not limited by the connecting portion, but solely by the shape of the base body, wherein by means of the side portions at the axial end portions of the base body, it can be determined how far the articulation members can be moved relative to one another until they touch.

In a preferred embodiment, the through-opening can be designed for the passage and defined accommodation of steering means, in particular steering cables or steering wires. For this purpose, molded parts can be formed from the base body, which can serve as guide elements for the steering means. In particular, the molded parts can be at least partially connected to the base body and form a defined guide channel within the inner cross section of the base body.

In a preferred embodiment of the deflection mechanism, a second connecting portion of one of the adjacent articulation members can be designed resiliently so that the keyed connection to the first connecting portion of the articulation member is established by a resilient deflection. This allows for easy assembly.

In a preferred embodiment of the deflection mechanism, the second connecting portion can at least partially encompass the extension. For example, the extension can be encompassed by the second connecting portion within a range of 60% to 90%, in particular 70% to 80%, if the second connecting portion has a fork shape. If the second connecting portion is formed as a type of tab with a recess, the second connecting portion can encompass the extension around the entire circumference.

In a preferred embodiment of the deflection mechanism, the second connecting portion can have a recess complementary to the extension so that a guided articulated connection is formed. The recess can be formed as a round through-opening in the second connecting portion. In particular, the diameter of the through-opening is adapted to the diameter of the extension.

In a preferred embodiment of the deflection mechanism, the deflection mechanism can be designed for one-time use. This is achieved in particular by the fact that the individual articulation members of the deflection mechanism can be manufactured economically and without great expenditure of time. On the other hand, the connection of the articulation members to one another can be implemented with little expenditure of time by eliminating additional connecting means and can be implemented by simply clamping or locking the connecting portions. In so doing, the deflection mechanism can be mounted without tools.

One-time use, also termed single use, offers several advantages. On the one hand, sterility is ensured from the outset and, in particular, cross-contamination is prevented. Furthermore, there is no effort for cleaning as well as no repairing effort for damaged elements. In particular with endoscopes that are very fragile, damage can quickly arise. A deflection mechanism for one-time use therefore offers the advantage that repairs are unnecessary due to the economical production. In contrast to reusable endoscopes, disposable endoscopes are therefore discarded after use. Re-pair of damaged products can therefore be dispensed with. Furthermore, several surgical instruments are usually used in parallel. This means that a high level of availability must be provided in order to be able to use several deflection mechanisms simultaneously. If each deflection mechanism is economical per se, several deflection mechanisms can be kept on hand without representing a financial burden.

In a preferred embodiment of the method, a second connecting portion of a second articulation member can be resiliently deformed while being displaced over the extension until the second connecting portion with a complementary recess at least partially surrounds the extension. This is implemented in particular with a second connecting portion which is shaped as a kind of tab with a recess.

In a preferred embodiment of the method, a second connecting portion of a second articulation member can be designed to be fork-shaped and is resiliently deformed while being displaced around the extension until a complementary recess of the fork shape surrounds the extension in a keyed manner. The second connecting portion has in particular two legs which are guided symmetrically around the extension. During assembly, the legs are resiliently spread until a recess between the legs, which is formed as an undercut for the extension, reaches the extension. In such a state, the spread legs can be resiliently deformed back so that the second connecting portion can securely grip around the extension, and the two articulation members are connected to one another in a keyed and articulated manner.

The above embodiments and developments can be combined with each other as desired, if appropriate. Further possible embodiments, developments, and implementations of the invention also include combinations, which are not explicitly mentioned, of features of the invention described above or below with respect to the exemplary embodiments. In particular, a person skilled in the art will also add individual aspects as improvements or additions to the particular basic form of the present invention.

### BRIEF DESCRIPTION OF THE DRAWINGS

The present invention is explained in greater detail below with reference to the exemplary embodiments shown in the schematic figures of the drawing. In the figures:

FIG. 1 is an isometric view of a deflection mechanism;

FIG. 2 is a detailed view from FIG. 1;

FIG. 3 is a further detailed view of the embodiment of FIG. 2;

FIG. 4 is a sectional view through the embodiment according to FIG. 2;

FIG. 5 is a further view of the embodiment of FIG. 2;

FIG. 6 is a plan view of another embodiment of a deflection mechanism;

FIG. 7 is an isometric view of the embodiment according to FIG. 6;

FIG. 8 is a sectional view through a deflection mechanism according to FIG. 7; and

FIG. 9 is a further view of the embodiment of FIG. 7.

The accompanying figures of the drawing are intended to provide a further understanding of the embodiments of the invention. They illustrate embodiments and, in conjunction with the description, serve to explain principles and concepts of the invention. Other embodiments and many of the advantages mentioned are shown in the drawings. The elements in the drawings are not necessarily shown to scale.

In the figures of the drawing, identical, functionally equivalent, and identically acting elements, features, and components are each provided with the same reference signs, unless other-wise specified.

### DETAILED DESCRIPTION

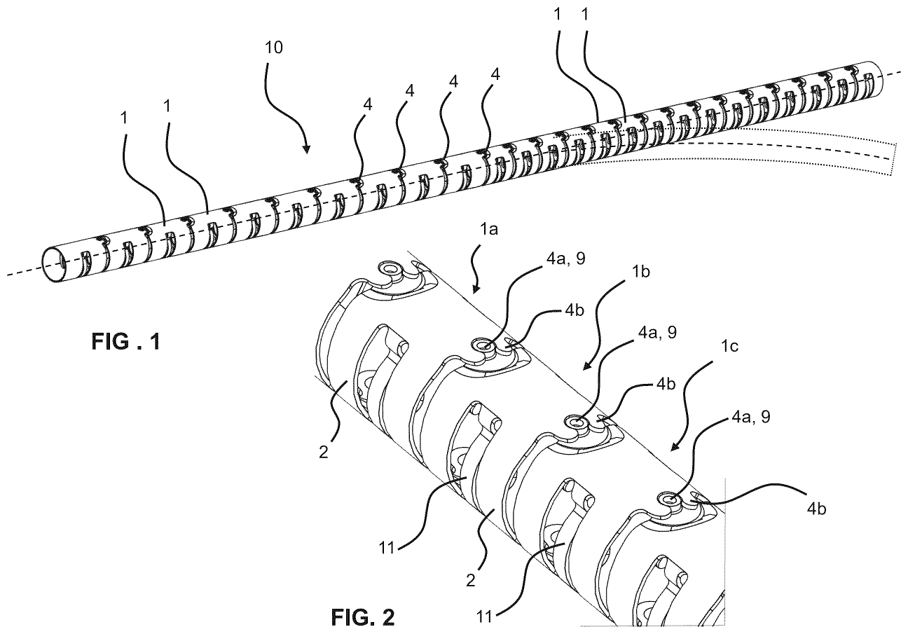

FIG. 1 shows an isometric view of a deflection mechanism 10. The deflection mechanism 10 is formed from several articulation members 1, which are connected to one another via connecting portions 4. As a result, the deflection mechanism 10 can be formed in an articulated manner so that it can be deflected from a longitudinal axis, as shown by way of example by the downwardly inclined and dashed view. The deflection mechanism 10 can have articulation members 1 of different lengths, whereby the radius of a deflection or a rotational movement can be influenced.

FIG. 2 shows a detailed view from FIG. 1. Several articulation members 1a, 1b, 1c are shown that each have a base body 2. The base body 2 is in particular tubular or sleeve-shaped so that a through-opening 3 can be formed. Furthermore, molded parts 11 can be partially cut out of the base body 2 and deformed in such a way that further guide channels are formed within the through-opening 3. The guide channels can be used in particular for the passage of a steering mechanism.

The illustrated articulation members are shown in a connected state, wherein the connecting portions 4a, 4b of adjacent articulation members are connected to one another. In the illustrated embodiment, the first connecting portions 4a each have an extension 9 which defines a joint axis 14, shown in FIG. 8.

In this embodiment, the second connecting portions 4b are designed to be fork-shaped, wherein the keyed and articulated connection is implemented by encompassing the extension 9 of the adjacent articulation member.

The connecting portions 4a, 4b of adjacent articulation members 1 are in particular aligned parallel to one another and/or formed complementarily to one another.

The articulation members touch one another at the connecting portions 4 only in the region of the first connecting portion 4a, wherein the remaining axial end portion of the articulation member 1a is arranged at a distance from the axial end portion of the articulation member 1b. As a result, a deflection from the longitudinal axis 7 can be achieved since the articulation members 1a, 1b, 1c can perform a rotational movement relative to one another. The extension 9 serves in particular to guide the rotational movement.

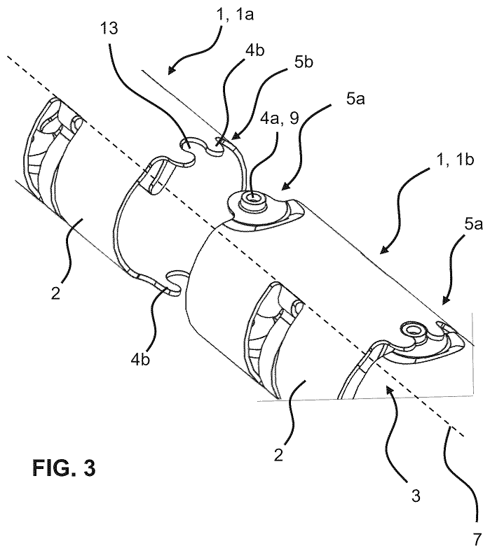

FIG. 3 shows a further detailed view of the embodiment according to FIG. 2. The articulation members 1 are shown before being pushed together. The second connecting portion 4b has two legs at a distance from one another, whereby the fork shape is formed. A recess 13 is formed between the legs, which is dimensioned as an undercut in particular to accommodate the extension 9.

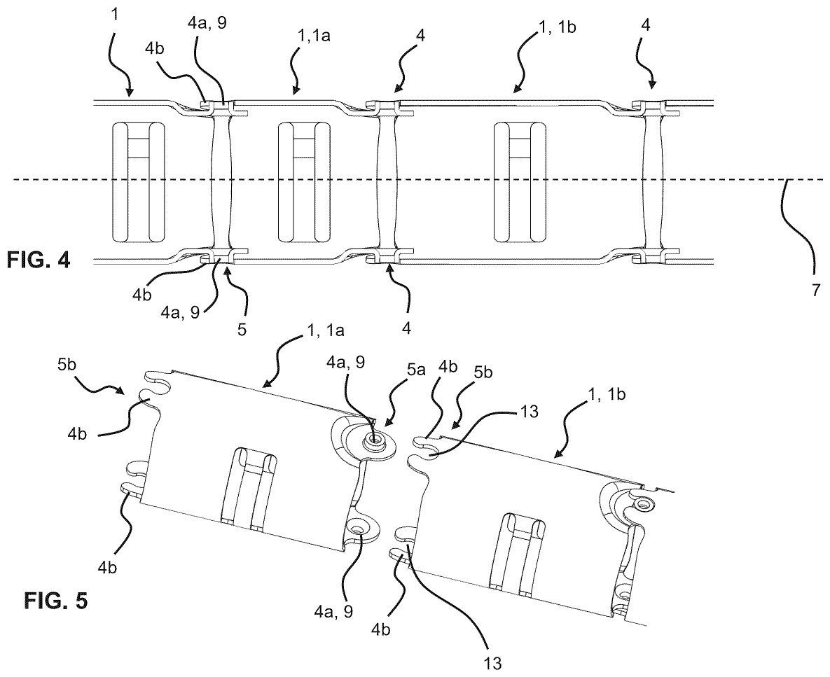

FIG. 4 shows a sectional view through an embodiment according to FIG. 2. In the sectional view it can be seen how the second connecting portion 4b encloses the first connecting portion 4a in the region of the extension 9. The extension 9 can assume the function of a connecting means such as a rivet connection or a connection by means of the bolt, with the advantage that the extension 9 is formed integrally with the articulation member 1.

The extension 9 is aligned in a direction transverse to the longitudinal axis 7 so that the extension 9 can be connected in one plane to a type of tab of the first connecting portion 4a and can enter into engagement in a plane different therefrom with a second connecting portion 4b of the adjacent articulation member 1. The two described planes are arranged offset from one another, in particular in the radial direction of the base body 2. This allows a robust joint connection to be implemented which can be designed in a similar way to a rivet connection.

The through-opening 3 is not affected by the extension 9. In contrast to a rivet connection or a connection with a bolt, the extension 9 advantageously does not protrude into the inner cross section of the through-opening 3, whereby an almost constant through-opening 3 can be provided throughout, which in particular has planar surfaces on the connecting portions 4a, 4b.

FIG. 5 shows a view of an articulation member 1. It can be seen that differently shaped connecting portions 4a, 4b are implemented at two axially opposite end portions 5a, 5b. In the left region of the articulation member 1a, the axial end portion 5b is provided on which a second connecting portion 4b is formed in two opposite regions. Each of the connecting portions 4b is designed to be fork-shaped so that an opening, in particular an undercut, is formed between two symmetrically designed legs. In the right region of the articulation member 1a, the axial end portion 5a is formed, wherein an extension 9 is formed in each of two opposite regions. The projections 9 are also formed mirrored to one another.

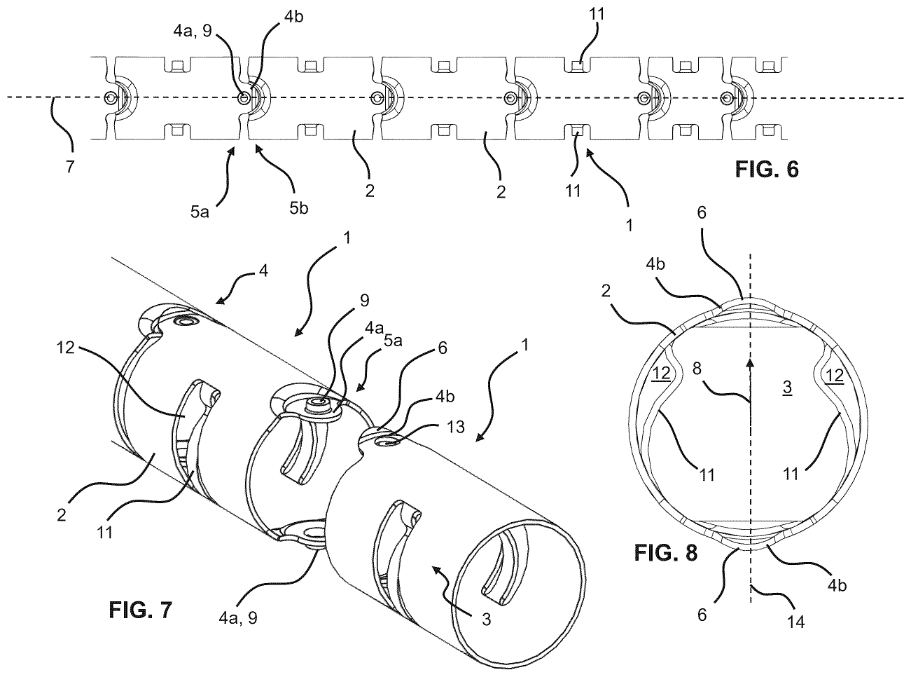

A further embodiment is shown in FIGS. 6 to 9. In this embodiment, the second connecting portion 4b is not fork-shaped, but rather a type of tab with a recess 13. The recess 13 can be formed as a through hole, in particular with a round cross section. In particular, the recess 13 is adapted in size and shape to the size and shape of the extension 9.

The second connecting portion 4b is therefore resiliently deflectable, in particular in this radial direction 8. This can be achieved, for example, by shaping it as a type of tab that projects beyond the base body 2 in the direction of the longitudinal axis 7 or protrudes beyond it. The second connecting portion 4b can also be resiliently deflectable transversely to a radial direction 8, i.e., in the circumferential direction. This can be achieved, for example, by shaping it in the manner of a fork that projects beyond the base body 2 in the direction of the longitudinal axis 7 or protrudes beyond it.

A guide contour 6 can be provided on the second connecting portion 4b, which supports the second connecting portion 4b in lifting itself over the extension 9 when the two articulation members 1 are pushed into one another. The guide contour 6 can be designed as a type of wing or wing tab. The guide contour 6 can be aligned in the direction in which the extension 9 is also aligned. The guide contour 6 therefore helps to allow an ergonomic and simple pushing of articulation members 1 into one another, for example in order to mount a deflection mechanism 10 on site.

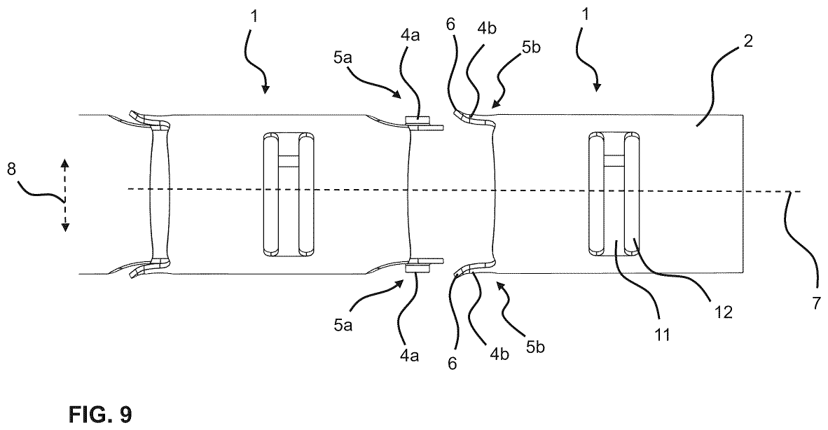

As can be seen in FIG. 9, the extension is at least partially aligned in the radial direction 8 of the base body 2. This is implemented regardless of the embodiment. This allows, for example, a joint axis 14 as shown in FIG. 8 to be implemented, which runs through a center of the base body 2.

Regardless of the embodiment, the articulation members 1 can be manufactured or cut out from a complete tube. This can be done in particular by laser cutting, plasma cutting, water jet cutting, punching or similar. Through a forming process, the extensions can be formed integrally with the base body 2. Alternatively, the extension 9 can be welded on. Other manufacturing methods are also conceivable. Since the tube consists in particular of a metal material, the second connecting portion 4b in particular is resiliently formed so that the articulation members 1 can be pushed into one another, wherein a connection can be established by a resilient deformation.

The deflection mechanism 10 can be used, for example, for different surgical instruments, in particular for catheters as well. The deflection mechanism 10 is designed in particular for small diameters of the articulation members 1. In particular, this allows diameters of 2 mm to 5 mm, in particular 3 mm, preferably less than 3 mm, to be implemented. In particular, diameters of less than 2 mm can be achieved.

The deflection mechanism 10 can, for example, also be mounted directly on site since mounting can be achieved by simply locking or threading the individual articulation members 1.

The deflection mechanism 10 or the articulation members 1 are particularly suitable for one-time use, i.e., for a single-use application, since they can be manufactured economically and connected to one another in a time-saving manner.

A plurality of embodiments and combinations thereof represent aspects of the disclosed invention, including, but not limited to, the following variations.

An articulation member (1) for or in a deflection mechanism (10) at a distal end of a surgical instrument, in particular an endoscope, having a base body (2) which has a through-opening (3) along a longitudinal axis (7) so that a partial portion of a working channel of the surgical instrument can be formed with the through-opening (3) or can be guided through the through-opening; and at least one first connecting portion (4a) which is arranged on a first end portion (5a) of the base body (2), wherein the connecting portion (4a) has an extension (9) at least partially aligned in a radial direction (8) of the base body (2) which is designed to be connected to a connecting portion (4) of a further articulation member (1) so that a keyed and articulated connection can be formed between two articulation members (1).

The articulation member (1) previously described, characterized in that a second connecting portion (4b) is arranged at a second end portion (5b) opposite the first end portion (5a), wherein the second connecting portion (4b) is designed resiliently so that the keyed connection to a connecting portion (4) of a further articulation member (1) can be established by a resilient deflection.

An articulation member (1) described above, characterized in that the second connecting portion (4b) is resiliently deflectable transversely to the radial direction (8) and/or in the radial direction (8).

An articulation member (1) described above, characterized in that the second connecting portion (4b) has a guide contour (6) which is arranged on the connecting portion (4b) at an end facing away from the base body (2) and allows for a guided resilient de-formation of the second connecting portion (4b) in the radial direction (8).

An articulation member (1) described above, characterized in that the second connecting portion (4b) is designed to be fork-shaped, wherein the keyed and articulated connection can be formed by encompassing the extension (9) of a further articulation member (1).

An articulation member (1) described above, characterized in that the second connecting portion (4b) has a recess (13) complementary to the extension (9).

An articulation member (1) described above, characterized in that two first connecting portions (4a) are provided on the first end portion (5a), which are in particular formed opposite one another on a cross section of the first end portion (5a) and/or mirrored to one another.

An articulation member (1) described above, characterized in that two second connecting portions (4b) are provided on the second end portion (5b), which are in particular formed opposite one another on a cross section of the second end portion (5b) and/or mirrored to one another.

An articulation member (1) described above, characterized in that the extension (9) is designed for articulated engagement with a connecting portion (4) of the further articulation member (1) on a predetermined articulation axis (14).

An articulation member (1) described above, characterized in that the opening (12) is designed for the passage and defined accommodation of steering means, in particular steering cables or steering wires.

A deflection mechanism (10) for alignment, in particular for tool alignment and/or for alignment of a lighting, rinsing and/or an imaging unit, at a distal end of a surgical instrument, in particular an endoscope, having: a plurality of articulation members (la, 1b) according to any of claims 1 to 10, wherein the articulation members (la, 1b) are connected to one another in a keyed and articulated manner such that an articulated working channel is formed through the through-openings (3) of the surgical instrument, or a flexible working channel can be passed through the through-openings (3).

The deflection mechanism (10) described above, characterized in that a second connecting portion (4b) of one of the adjacent articulation members (1b) is designed resiliently so that the keyed connection to the first connecting portion (4a) of the articulation member (la) is established by a resilient deflection.

A deflection mechanism (10) described above, characterized in that the second connecting portion (4b) at least partially encompasses the extension (9).

A deflection mechanism (10) described above, characterized in that the second connecting portion (4b) has a recess (13) complementary to the extension (9) so that a guided articulated connection is formed.

A deflection mechanism (10) described above, characterized in that the deflection mechanism (10) is designed for one-time use.

A method for producing a deflection mechanism (10) for alignment, in particular for tool alignment and/or for alignment of a lighting, rinsing and/or an imaging unit, at a distal end of a surgical instrument, in particular an endoscope, comprising the steps of: providing a plurality of articulation members (1) according to any articulation member described above, at least partially pushing adjacent articulation members (la, 1b) into one another, wherein connecting portions (4) of adjacent articulation members (1a, 1b) are displaced over one another until an extension (9) of a first connecting portion (4a) at least partially aligned in a radial direction (8) of the base body (2) engages in a connecting portion (4) of a second articulation member (1b) so that a keyed and articulated connection of the articulation members (1a, 1b) is produced.

The method described above, characterized in that a second connecting portion (4b) of a second articulation member (1b) is resiliently deformed while being displaced over the extension (9) until the second connecting portion (4b) with a complementary recess (13) at least partially surrounds the extension (9).

A method described above, characterized in that a second connecting portion (4b) of a second articulation member (1b) is designed to be fork-shaped and is resiliently deformed while being displaced around the extension (9) until a complementary recess (13) of the fork shape surrounds the extension (9) in a keyed manner.

Although the present invention has been fully described above with reference to preferred exemplary embodiments, it is not limited thereto, but can be modified in a variety of ways.

### LIST OF REFERENCE SIGNS

1 Articulating member
2 Base body
3 Through-opening
4 Connecting portion
5 End portion
6 Guide contour
7 Longitudinal direction
8 Radial direction
9 Extension
10 Deflection mechanism
11 Molded part
12 Opening
13 Recess
14 Joint axis

1. An articulation member for or in a deflection mechanism at a distal end of a surgical instrument, having:
a base body which has a through-opening along a longitudinal axis so that a partial portion of a working channel of the surgical instrument can be formed with the through-opening or can be guided through the through-opening; and
at least one first connecting portion which is arranged on a first end portion of the base body, wherein the connecting portion has an extension at least partially aligned in a radial direction of the base body which is designed to be connected to a connecting portion of a further articulation member so that a keyed and articulated connection can be formed between two articulation members.

2. The articulation member of claim 1, wherein a second connecting portion is arranged at a second end portion opposite the first end portion, wherein the second connecting portion is designed resiliently so that the keyed connection to a connecting portion of a further articulation member can be established by a resilient deflection.

3. The articulation member of claim 2, wherein the second connecting portion is resiliently deflectable transversely to the radial direction or in the radial direction.

4. The articulation member of claim 3, wherein the second connecting portion has a guide contour which is arranged on the connecting portion at an end facing away from the base body and allows for a guided resilient deformation of the second connecting portion in the radial direction.

5. The articulation member of claim 2, wherein the second connecting portion is designed to be fork-shaped, wherein the keyed and articulated connection can be formed by encompassing the extension of a further articulation member.

6. The articulation member of claim 3, wherein the second connecting portion is designed to be fork-shaped, wherein the keyed and articulated connection can be formed by encompassing the extension of a further articulation member.

7. The articulation member of claim 2, wherein the second connecting portion has a recess complementary to the extension.

8. The articulation member of claim 6, wherein the second connecting portion has a recess complementary to the extension.

9. The articulation member of claim 1, wherein two first connecting portions are provided on the first end portion, that are formed opposite one another on a cross section of the first end portion or mirrored to one another.

10. The articulation member of claim 1, wherein two second connecting portions are provided on the second end portion, which are in particular formed opposite one another on a cross section of the second end portion or mirrored to one another.

11. The articulation member of claim 1, wherein the extension is configured for articulated engagement with a connecting portion of the further articulation member on a predetermined articulation axis (14).

12. The articulation member of claim 1, wherein the opening is designed for the passage and defined accommodation of steering means, such as steering cables or steering wires.

13. A deflection mechanism for alignment, in particular for tool alignment and/or for alignment of a lighting, rinsing and/or an imaging unit, at a distal end of a surgical instrument, having:
a plurality of articulation members, each articulation member having a base body which has a through-opening along a longitudinal axis so that a partial portion of a working channel of the surgical instrument can be formed with the through-opening or can be guided through the through-opening, and at least one first connecting portion which is arranged on a first end portion of the base body, wherein the connecting portion has an extension at least partially aligned in a radial direction of the base body which is designed to be connected to a connecting portion of a further articulation member so that a keyed and articulated connection can be formed between two articulation members; and
wherein the articulation members are connected to one another in a keyed and articulated manner such that an articulated working channel is formed through the through-openings of the surgical instrument, or a flexible working channel can be passed through the through-openings.

14. The deflection mechanism of claim 13, wherein a second connecting portion of one of the adjacent articulation members is designed resiliently so that the keyed connection to the first connecting portion of the articulation member is established by a resilient deflection.

15. The deflection mechanism of claim 14, wherein the second connecting portion at least partially encompasses the extension.

16. The deflection mechanism of claim 15, wherein the second connecting portion has a recess complementary to the extension so that a guided articulated connection is formed.

17. The deflection mechanism of claim 13, wherein the deflection mechanism is configured for one-time use.

18. A method for producing a deflection mechanism for alignment, in particular for tool alignment and/or for alignment of a lighting, rinsing and/or an imaging unit, at a distal end of a surgical instrument, comprising the steps of:
providing a plurality of articulation members, each articulation member having a base body which has a through-opening along a longitudinal axis so that a partial portion of a working channel of the surgical instrument can be formed with the through-opening or can be guided through the through-opening, and at least one first connecting portion which is arranged on a first end portion of the base body, wherein the connecting portion has an extension at least partially aligned in a radial direction of the base body which is designed to be connected to a connecting portion of a further articulation member so that a keyed and articulated connection can be formed between two articulation members; and
at least partially pushing adjacent articulation members into one another, wherein connecting portions of adjacent articulation members are displaced over one another until an extension of a first connecting portion at least partially aligned in a radial direction of the base body engages in a connecting portion of a second articulation member so that a keyed and articulated connection of the articulation members is produced.

19. The method of claim 18, wherein a second connecting portion of a second articulation member is resiliently deformed while being displaced over the extension until the second connecting portion with a complementary recess at least partially surrounds the extension.

20. The method of claim 18, wherein a second connecting portion of a second articulation member is designed to be fork-shaped and is resiliently deformed while being displaced around the extension until a complementary recess of the fork shape surrounds the extension in a keyed manner.
